(19) United States
(12) Patent Application Publication
(10) Pub. No.: US 2020/0345394 A1
(43) Pub. Date: Nov. 5, 2020

(54) Protector for a Trans-Corneal Incision

(71) Applicant: Richard B. Foulkes, Lombard, IL (US)

(72) Inventor: Richard B. Foulkes, Lombard, IL (US)

(21) Appl. No.: 16/863,046

(22) Filed: Apr. 30, 2020

Related U.S. Application Data
(60) Provisional application No. 62/841,493, filed on May 1, 2019.

(51) Int. Cl. A61B 17/34; A61F 9/007

(52) U.S. Cl. CPC A61B 17/3423; A61B 2017/00889; A61F 9/007

(57) ABSTRACT

A protector is engineered to protect corneal tissue during intraoperative procedures. The protector is a temporary passageway into and optionally through the cornea. The sleeve portion of the protector can be more rigid or less rigid depending on a procedure or surgeon preference. The sleeve is described herein in terms of two examples. The first example is a polymer fabric sleeve that expands and is secured into an incision that will then be the passage for all instruments entering the eye. It will also seal the eye from backflow of liquids and tissue out of the passageway. The second example is a relatively rigid tube with an external bladder that is expandable into the incision to secure it from movement and acts as a valved passage into the eye.

This application claims the benefit of U.S. Provisional Patent Application No. 62/841,493, filed May 1, 2019, which is incorporated by reference herein in its entirety.

The present invention relates to an insertable sleeve or tube for use in trans corneal procedures to protect the cornea and other eye layers during the procedures as well as sealing the eye from rapid outflow. The sleeve or tube is inserted into an incision and protects the incision and eye layers inside from disturbance or damage. The sleeve or tube are also self-sealing and prevent unintended outflow from the eye.

### BACKGROUND

Intraocular surgery in the anterior portion of the eye has evolved over time. Initially an eye would be opened for cataract surgery with a large 10+ mm opening at the scleral corneal border then sutured closed after the procedure was complete. With the advent of a method of lens extraction using vacuum and ultrasound and flexible intraocular lenses, the incision size was reduced and the entry area moved from the scleral corneal border to the clear cornea.

Currently clear cornea is the predominant method for most procedures involving the anterior segment of an eye. In this method a blade is used to create a self-sealing incision. Though there are many small variants on the technique in general, the surgeon begins the entrance at the scleral corneal border bringing the path parallel to the surface for the initial 2-3 mm and then angling into the anterior chamber. Over time this incisional dimension is believed to give the best balance of freedom to work in the eye, control of outflow during surgery and self-sealing without need or a suture closure at the end of the procedure. The dimensions of the corneal opening can vary but a square or somewhat rectangular incision is the normal result. With a blade there is often significant variation with the length often being longer or shorter than intended. If the incision length is longer, the angle of instrumentation down to the cataract is greater and the cornea must be depressed under the incision which can lead to distortion or inability to optimally use instruments in the eye to access the cataract under the incision. If the incision length is too short, the incision may not be self-sealing, and unintentional iris extrusion during or after the procedure is possible from expulsion of water out of the incision. As the amount of stroma is less, forgiveness of the physical trauma to the stroma is greater and these short incisions are more likely to leak and give way post operatively. A suture may be needed to secure the incision in the immediate postoperative period.

The cornea is composed of a skin layer based on a tough thin layer called Bowman's. Below the Bowman's layer is a lamellar tissue known as the stroma. It is crosslinked and more adherent to itself near the surface and becomes more hydrated and less dense near the interior of the eye. The inner layer of the cornea is an important tissue based on a membrane known as Descemet's membrane. The Descemet's membrane includes cells that are pumping cells and are critical to corneal function. These cells are called endothelial cells. If these cells are injured or dislodged from their base, the cornea above them will thicken from fluid. Normal thickness of a cornea is variable but generally is 540 um centrally and 6-700 um at the corneal scleral rim where the incision is created. Without endothelial pumping, the cornea will become opaque from swelling of fluid and the thickness can double from normal. To protect these cells during cataract surgery a viscoelastic hyaluronic gel is placed into the anterior chamber that coats these cells. The significant fluid flow and ultrasound used could injure them. If the surgeon contacts the endothelial cells with an instrument the Descemet's membrane can be dislodged creating a ‘flap” of these cells which are now not attached, and the eye will thicken above. Often after completion of modern cataract extraction, a gap is seen where the inner opening of the eye has been stretched or the devices have pushed back a small section of the endothelium. If this occurs near the center of the cornea the results can be minor to major alterations of vision from the distortion created by the regional swelling. The total count of cells varies across the cornea but humans are born with 2500-3500 cells per square mm and the count may fall throughout a lifetime. When the number falls below 1000 and especially near 500 the cornea will have an excess of fluid which leads to micro cysts under the surface known as “bullous keratopathy” and corneal fogging from the water altering the architecture of the layers.

During cataract surgery, a corneal incision will have several instruments inserted through the incision to first remove a cataract and then insert an intraocular lens. The primary instrument, named a phacoemulsifier, uses ultrasound that is generated in the handle and then directed into the metal tip directionally to soften and then allow vacuum extraction of lens material. When ultrasound is required to soften the cataract a small amount of heat is generated. To modulate this the device has a silicone sleeve and water is flowing around the metal needle to equal the flow out of the eye as lens material is vacuumed out. Without this and sometimes with it heat can injure the tissue causing a mild to severe burn in the cornea. Severe burns are rare today and minor inflammation is likely more common especially in cases requiring higher need for ultrasound. The tip of the phaco instrument is metal, sharp and either straight or angled. It must pass through an incision tunnel to begin the surgery and is moved in and out to manipulate the lens and capture lens fragments. There is significant flow around the needle tip out of the eye which hydrates the tissue of the stroma and especially the less crosslinked inner cornea. This increases the risk of weakening the attachment of Descemet's membrane. The lamellar tissue of the cornea is layered and the devices sliding in and out of the eye separate the layers from frictional and hydrational effects. Following lens removal, another instrument is introduced to remove the softer cells that surround the cataract and that are adherent to the lens capsule and then are reintroduced after the IOL is placed. To remove the “cortex” or the cells adherent to the delicate lens capsule the tip has a smaller opening and it is angled with stripping motion to “peel” the cells off the capsule. There is more stretching and incisional stress in this step of the procedure as the tip is angled to reach the area adjacent and underneath the incision. With both of these instruments the sleeve is in constant contact with the endothelial edge of the incision sliding in and out of the eye, and as it is narrower than the phaco instrument a significant amount of backflow will pass along the outside as pressure builds in the eye. It is often after withdrawal of this device that iris tissue will flow with it out through the incision. Finally, a tube containing the intraocular lens (IOL) is inserted into the incision to push the IOL into the eye. The width of this tube is variable by the style, volume and flexibility of the implant. Larger lenses are thicker and will require a larger tube. This step is often the incision size limiting point. The current variability is 2.2 mm to 3.0 mm on average. 2.4 mm to 2.6 mm is required to complete most cataract surgery. As these instruments are round it should be noted that the diameter of the inserted instruments is not the incisional size. The tube width or diameter at 2.4 incision cannot be more than 1.5 mm. This tip is angled through the incision which is often just large enough to pass it. The tube is made of a distensible plastic. It can catch the lip of the inner part of the incision and strips back a small area of the Descemet's membrane and endothelial cells. Though it has not been studied it is likely a point where this particular incisional trauma occurs. To balance the needs of lens insertion, minimal back flow during surgery and self-sealing at the end of the case have been the guiding issues surrounding incision size and care. Indeed, larger incisions would likely be more commonly used if the incision was secure during and after surgery.

A cataract surgery typically also uses a second smaller incision of approximately 1 to 1.5 mm that is made laterally to the primary incision. This incision receives the most amount of instrumentation. It is often referred to as the “sideport” incision. This second incision is then used to push fluids and gels into the eye, manipulate the lens using a hooked instrument to “chop” the cataract, position the IOL and experience the largest amount of out flow during the surgery as the primary incision is more occluded by larger instruments and their sleeves. Any need to work laterally into the eye uses this incision. As the surgeon angles thin instruments through this opening the internal pressure of the eye is relieved and water can be observed flowing rapidly. If 150 cc of balanced saline solution is required in a cataract surgery ⅓ to ½ of this will flow from this incision. Again, its inner edge often remains gaped after the procedure though rarely recognized due to its small size. It often has leakage at the conclusion of the surgery from stretching and manipulation. The maintenance of a stable anterior posterior environment is essential in cataract surgery as the vacuuming tips are working within less than a mm of the lens capsule. If this capsule is inadvertently torn the vitreous held back by the capsule will come forward and lens particles can fall into the back of the eye. The intraocular lens may not be well centered and it generally cannot be placed into its normal position inside the capsular bag as it would not be supported. The lens power must be reduced and the lens is placed on top of the capsule if possible. These patients are at higher risk of post op issues like cystoid macular edema and endophthalmitis. Maintaining a stable anterior chamber is about reducing sudden motion which is normally from a sudden reduction in the internal pressure as water ejects from an incision. Sealing these incisions therefore stabilizes the eye, and leads to a reduction in the total amount of fluid needed to complete the cataract.

At the end of a procedure water is injected into the cornea to expand the tissue laterally and tighten the pocket to reduce fluid egress and ingress post operatively. The more the incision has been manipulated, stretched and stripped, the poorer this is likely to work in the minutes to hours after the patient is discharged. The injected fluid into the tissue resolves within minutes of the patient leaving the OR and if wound leakage is more likely in an eye that has had a large amount of instrumentation. There is evidence that in longer surgeries and surgeries where complication led to more instrumentation that a higher rate of endophthalmitis can occur. This is a rare but sight threatening infection inside the eye. These wounds can be seen years later as with haze and scarring from instrumentation trauma.

Careful examination of these incisions will demonstrate swelling and endothelial cell loss under and around these incisions. A gap or tear in the Descemet's membrane and the endothelial cells adherent to it is a ubiquitous finding. In many corneas the area can be observed to have thickening and fogginess associated with endothelial dysfunction. The longer term sequelae are less common. A persistent area of edema or thickening creates a foreign body or “dry eye” sensation. The thickening of the cornea may create a light or “fog” visual effect from the cornea being swollen laterally.

There are also many reasons for the iris or colored part of the eye to be traumatized during cataract surgery. The common cause of injury is from this thin friable tissue flowing back out of the eye through the incision. This is known as iris extrusion. The pressure in the eye is elevated by fluid flowing into the eye and on removal of an instrument a rush of water carries the iris through the incision. The surgeon must decrease the pressure inside the eye and carefully move the iris back into the eye. When this happens, the pigment covering this tissue is dislodged and light can now pass through the tissue leading to glare and secondary images. This is made worse when the patient feels pain as this tissue is sensitive and when it is squeezed out of the incision it induces further squeezing. Using viscoelastic and patience, the surgeon can often regain control and finish the case. Unfortunately, the iris is now injured. The pigment is stripped and the pupil may not return to a normal round shape. A short incision and can occur in any case even without the pathologies described. And as the short incision is less effective at sealing the eye, the likelihood of extrusion is increased. As the surgeon is aware of this, currently there is no effective method to assure that extrusion will not occur. A glare effect from light passing through the damaged area, a distorted pupil or both is the result of this uncontrolled flow out of the incision. The dependence on the random hand-made incision is a weakness of modern cataract surgery for this reason alone. Backflow out of the eye is a random and sight threatening event that occurs regularly with current technique and tools.

A thin iris is often light in color or has been weakened by drugs. A condition known as IFIS or Intraoperative Floppy Iris Syndrome is common and can be the result of the use of alpha1-antagonists for Benign Prostatic Hypertrophy as well as a myriad of other conditions. If a pupil is weak and thin, it can obscure visibility adequate for cataract extraction and require the use of hooks to capture the iris or a device extruded into the eye like a Malyugin ring. Often the dilation is adequate, but the risk of iris extrusion is so high that the ring is used to control the iris from extruding.

The insertion of an IOL is often the moment when the most stress is placed on the incision as this is the widest instrument used. It is likely that the stripping of the endothelium is occurring often in this step as the surgeon pushes against resistance to stretch the incision to get the nozzle of the inserter beyond the inner lip of the cornea.

### SUMMARY

It is an object of the present invention to overcome or reduce the challenges of intraocular eye surgery as explained above. The intent of these devices is to protect the cornea from instrumentation, to seal the eye to decrease uncontrolled outflow and to act as a conduit into the eye for treatments and monitoring.

Simple and inexpensive devices are described herein for protecting the corneal tissue from instrumentation and to seal the eye from uncontrolled outflow. As it is a sealed conduit into the eye other uses may be applied. These devices include sleeves or tubes that may be inserted into the eye during surgery. The devices provide a passage from an eye incision into the portion of the eye where a procedure is performed. The sleeve protects all of the various components of the eye that must be passed through by instruments during a procedure. A proximal end of the sleeve is anchored to the corneal incision to prevent the sleeve from being pushed into the eye. The distal end of the sleeve inside the eye is supported so that the sleeve is stable with instrumentation during a procedure. The device seals backflow as the sleeve is stretched against itself or a plate. The tube has a collapsible valve to prevent backflow.

In one example, a protector for a trans-corneal incision comprises a tubular sleeve that defines a width and length, wherein the sleeve has a proximal end and a distal end. The proximal end includes wings that extend outwardly from the width of the sleeve, and the proximal end is adapted to engage the perimeter of an ocular incision and the wings extend outside of the perimeter of the ocular incision to prevent the sleeve from being pushed into an eye. Wherein the distal end is adapted to extend into intraocular eye space in the direction of the length of the membrane. The tubular sleeve may be a flexible membrane. The protector may further comprise a frame that is connected to the sleeve and holds the sleeve in an expanded position to secure the sleeve in an eye. The tubular sleeve membrane may be multilayered and comprise a sealed bladder that is open to an exterior valve. The tubular sleeve membrane is expandable by injecting air or fluid into the sealed bladder in the membrane and through the valve. The sleeve may further comprise a plurality of small spikes that project outwardly from the outside of the sleeve for securement of the sleeve in an intraocular incision. In another example, the tubular sleeve may comprise a rigid tube. The rigid tube may have an expandable bladder mounted around an outside portion of the rigid tube. The sleeve may have a sealed inner layer of fluid therein in order to moderate heat transfer through the membrane wall. The sleeve may be colored or clear. The sleeve may be coated on its outside surface with an antibiotic material or an anti-inflammatory material. The sleeve may further comprise an expandable spring to actuate the expansion of the sleeve. The frame may comprise two arms connected by an arch, and the arch having raised elements on its posterior surface to secure to the flexible membrane.

### BRIEF DESCRIPTION OF THE DRAWINGS

FIG. 1 is a top view of a sleeve as described herein with a frame mounted therein.

FIG. 2 is a top view of the sleeve alone also shown in FIG. 1.

FIG. 3 is a perspective view of the sleeve shown in FIGS. 1 and 2.

FIG. 4 is a top view of an eye with a pair of sleeves mounted therein as described herein.

FIGS. 5 and 6 are top views of an eye as shown in FIG. 4 with examples of medical instruments shown passing through the sleeves.

FIGS. 7A and B are side and top views respectively of an example of a frame to be sued with a sleeve.

FIGS. 7C and D are perspective and side views respectively of a sleeve and frame combination.

FIGS. 8A and B are top views of a frame inside a sleeve with two examples of securing the distal end of the frame legs in the sleeve.

FIG. 9 is a perspective view of a further example of a sleeve as described herein.

FIG. 10 is a front view of the sleeve shown in FIG. 9.

FIG. 11 is a side view of the sleeve shown in FIGS. 9 and 10 with its bladder being uninflated.

FIG. 12 is a side view of the sleeve shown in FIGS. 9, 10 and 11 with its bladder being inflated.

FIGS. 13 and 14 are side views of another example of a sleeve as described herein showing a bladder in its inflated (FIG. 13) and uninflated (FIG. 14) conditions.

FIG. 15 is a front view of a sleeve having a valved orifice.

FIG. 16 is a top view of an eye with a pair of another example of sleeves mounted therein as described herein.

FIGS. 17 and 18 are perspective views of another example of a frame with an internal flat plate with its sleeve unattached and attached respectively.

FIG. 19 is a bottom perspective view of the frame shown in FIG. 17.

### DETAILED DESCRIPTION

The device described herein is engineered to protect corneal tissue during intraoperative procedures. The various design elements serve to seal the incision from backflow. In one example, a sleeve is stretched over the frame to create a seal when closed and allow passage of instrument of variable size. The sleeve is described herein in terms of two examples. Similar protector devices may be designed that are also covered by this disclosure. The first example is a polymer or fabric sleeve that expands and is secured into an incision that will then be the passage for all instruments entering the eye. It will also seal the eye from backflow of liquids and tissue out of the passageway. The second example is a relatively rigid tube with an external bladder that is expandable into the incision to secure it from movement and acts as a valve for passage into the eye without backflow.

The first example of a sleeve described herein is used in intraocular procedures to pass from outside the eye to inside the eye encompassing the width and length of an incision made to access the anterior chamber. The protector device includes a sleeve that enables the use of tools used in an intraocular procedure to pass into and out of the intraocular space without coming into direct contact with the tissue along its length. It effectively seals the eye eliminating inadvertent backflow and stabilizing the intraocular environment from collapse because it is stretched apart by a number of described devices. The sleeve is secured to the eye incision by several methods both externally and internally. The protector sleeve may be composed of elastic material that allows it to be expanded into the exact fit of the incision while sealing around instruments as they are inserted into the eye. These materials include latex, rubber, alginate polyacrylamide, a hydrogel, polymers, neoprene, fabric material like spandex, nitrile rubber or synthetic rubber, polyvinyl chloride and other known elastic materials. The sleeve is open externally by a securing method to allow for easy access. The distal end of the sleeve inside the eye will be stretched closed when an instrument is not passing through it. The sleeve effectively acts as an extension of the incision deep into the eye as it seals similar to the “pocket” like incision.

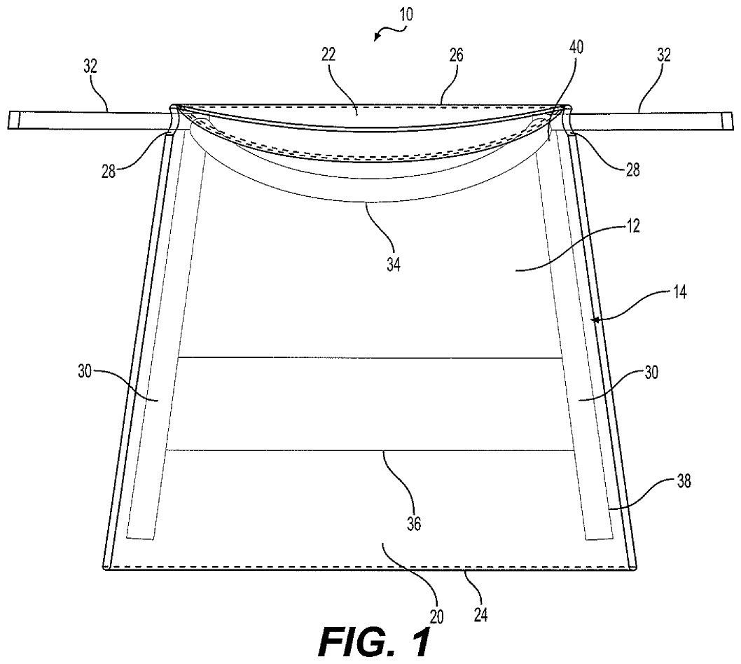

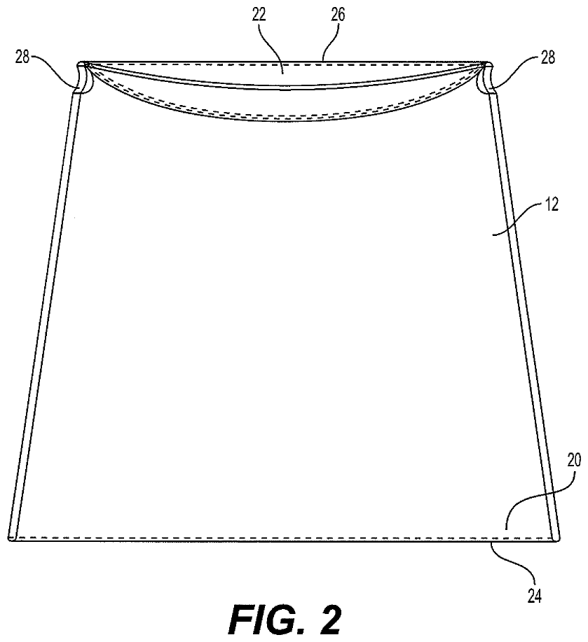

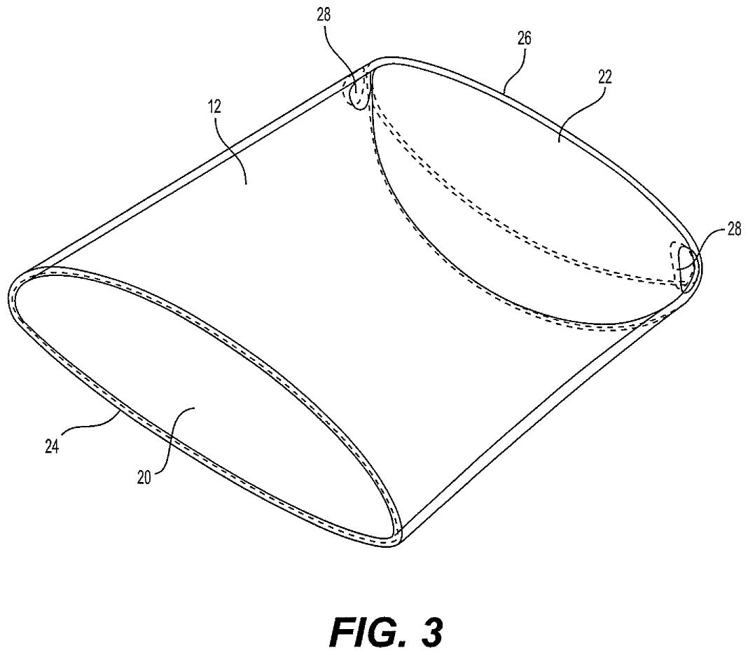

The first protector device, an example of which is shown in FIGS. 1-3, is composed of two basic components—a sleeve 12 that is secured to a frame 14. The sleeve 12 is a tube that has a proximal opening 26 and a distal opening 24. The frame 14 is able to expand and stretch the sleeve 12 to the length and width of an incision where the protector 10 may be placed. The expansion feature of the frame 14 may be enabled by several different methods to allow for adequate tension so that the sleeve 12 will be stable while instruments are pushed into and pulled out of the eye. The frame 14 must hold lateral tension and expand the sleeve in order to remain stable in the incision. In the simple protector 10 shown in FIG. 1, an external arch 34 is used to create separation and tension in an incisional area. The arch 34 is connected to two wings 32 that protrude laterally wider than a corneal incision to secure the protector 10 from being pushed into the eye and can be held when an instrument is withdrawn to prevent expulsion. The sleeve 12 will be stretched over the wings 32 and over the top of the arch 34. The wings 32 extend through holes 28 in the proximal end 22 of the sleeve 12 and thereby secure the sleeve externally to the frame 14 and the arch 32 provides an opening to begin easy passage through the proximal opening 26 of the sleeve and into and through the sleeve. The arms 30 have a proximal end 40 connected to the wings 32 and a distal end 38 near the distal end 20 of sleeve 12.

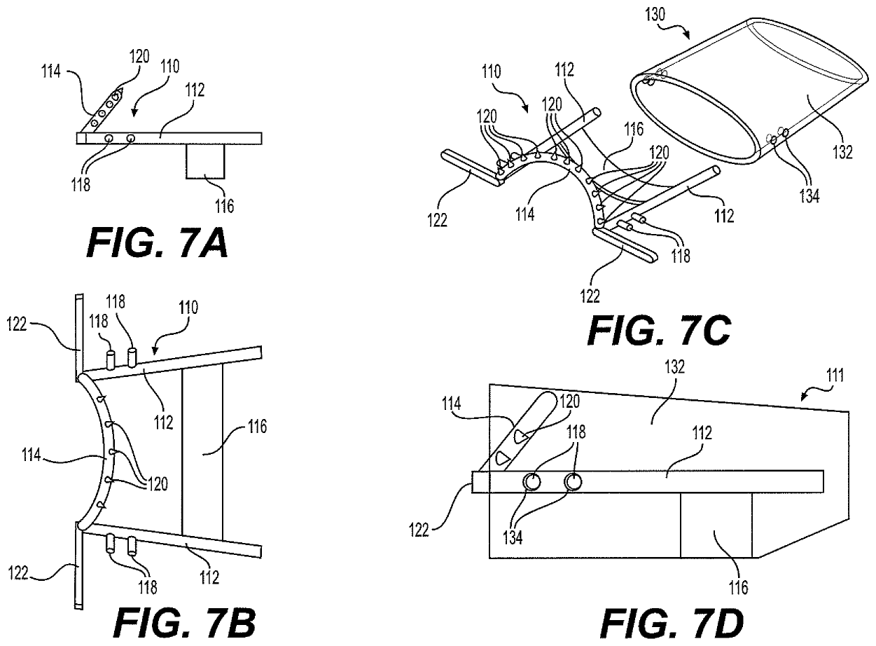

In another example of a protector 111, as shown in FIGS. 7A-D, arms 112 of the frame 110 extend into the eye and can have short pegs 118 extending outwardly to expand into the stroma within the first 2 mm of the incision. These pegs 118 are shown in an alternative example of a protector 111 as shown in FIGS. 7A-D. These pegs 118 also pass through holes 134 in sleeve 132 and would further, or alternatively, secure the sleeve from displacement in or out during use. In addition, further pegs 120 may be positioned facing externally along the arch 114 to also secure the elastic material of a sleeve 132 in use.

The expansion arms 30 (FIGS. 1-3) and 112 (FIGS. 7A-D) width (the distance between the respective arms) can be very thin to minimize incision expansion. The protector 10 and 111 can be inserted into the incision in an eye and then expanded to be secured therein. Expander mechanisms may include a spring-like circle that is squeezed together so that it expands into the incision, a ratchet, or a screw expander. These expanders are limited by wings used to limit the passage of the device and stabilize the sleeve which would be attached to the wings.

One method of expansion is a spreading of the sleeve 12 and 130 by using a flexible plastic to form the frame 14 and 110 that is molded and biased toward expansion and that is squeezed together by a surgeon for insertion and, when released, expands the sleeve into the width of the incision. The arch 34 and 114 is the expansive force holding the protector 10 and 111 in place at the incision. The distal end 20 of the sleeve 12 is wider than the proximal end 22 of the sleeve. Between the arms 30 and 112 of the protector 10 and 111 past the incision, a flat crossmember 36 and 116 with the V shape made of the same flexible plastic as the rest of the frame 14 and 110 will add further structure and expansive force to the device. The crossmember 36 and 116 may also assist in securing in place a patient's iris by bearing against it during a procedure. This crossmember 36 and 116 will have a thin extension which is a landing pad for instruments to guild them into the eye over the top of the sleeve. The sleeve 12 and 132 is designed to remain in place against movement of medical instruments through the sleeve both in and out during a procedure. The sleeve 12 and 132 can elongate as it passes over the external arch and folded back under the arch. As shown, a crown of pegs 120 or spikes may be added to secure the flap of sleeve 12 and 132 draping over the arch 34 and 114. One example of a sleeve 132 may have lateral holes 134 to be captured by the pegs 118 in the first 1-2 mm of the internal arms 112. No holes are necessary for this additional securing point of the sleeve. Also, the crossmember may not be necessary for securing the protector 10 and 111.

The size of the arch 34 and 114 would vary by incisional size. Before insertion the width may be 3 mm or greater for a primary incision version. Inserted the width across the arch 34 and 114 would be whatever the incision size determined. A common result would be 2.4-2.6 mm. This width would allow for passage of many instruments including an intraocular lens inserter.

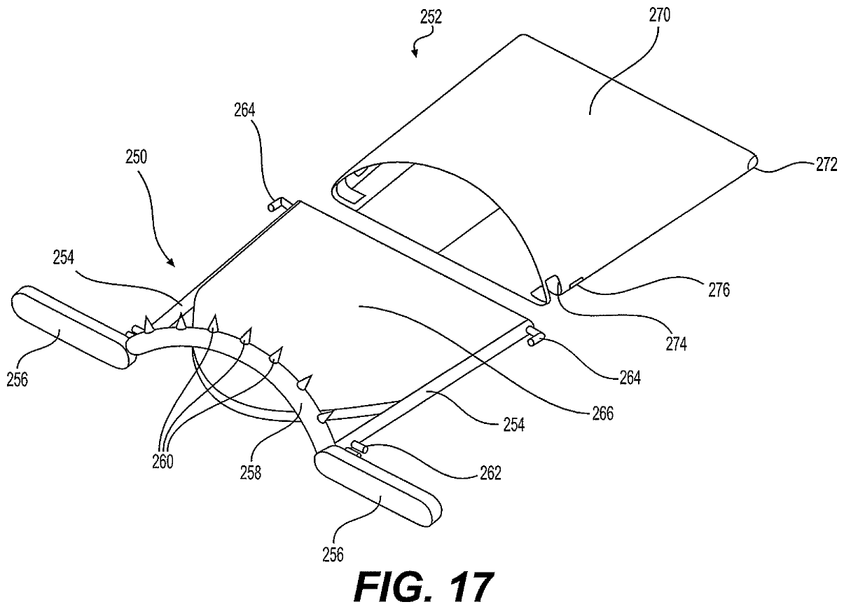

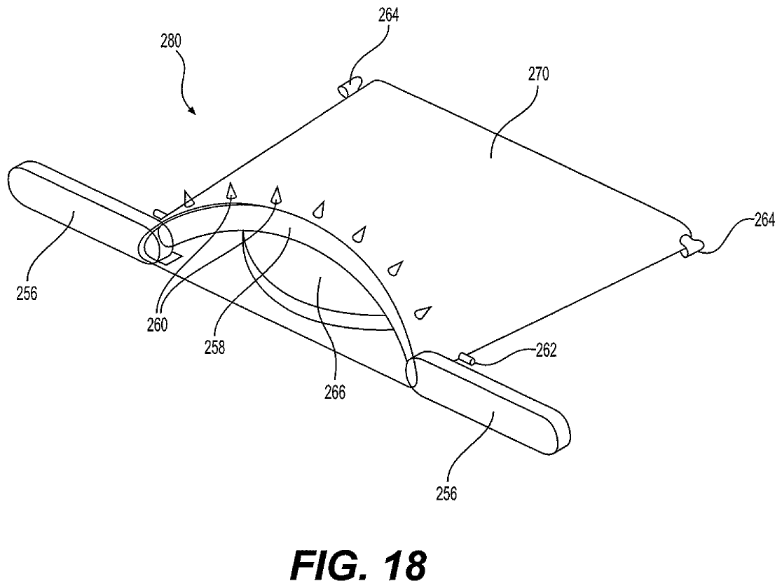

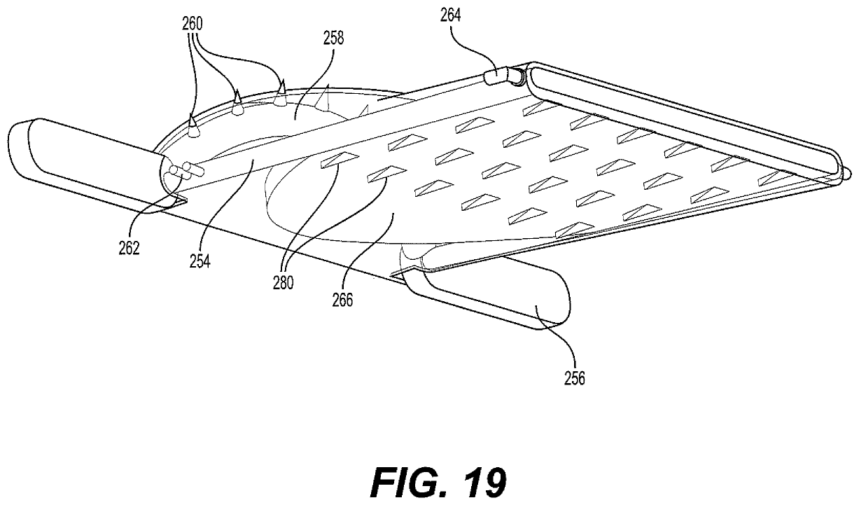

Another example of a protector 280 is shown in FIGS. 17-19 comprises a frame 250 and sleeve 252. The frame 250 include a pair of arms 254 connected to two wings 256 and a connecting arch 258 between the arms. The arms 254 have outwardly protruding pegs 262 at their proximal end and sleeve hooks 264 at their distal end. The arch 258 has upwardly protruding pegs 260 that engage the sleeve 252 and hold it secure to the arch 258. This example of a frame 250 includes a glide sheet 266 that is positioned between the arms 254. The glide sheet 266 is a thin sheet between the arms 254 that extends from the distal end of the arms toward the proximal end. If the glide sheet 266 extended through incision it would take up volume and require a larger incision. The flexible side arms 254 connected to the arch 258 takes the least amount of space and allows for an incision to be variable in width while filling it. The thin 0.1 to 0.15 mm arms 254 are separated by the arch as well as the thin glide sheet 266 are found in the incision. Optionally glide sheet 266 could advance to the incisional opening. As shown in FIG. 19, the glide sheet 266 may have a textured bottom as illustrated by spikes 280. The arch 258 can stretch to 3+ mm and the incision is 2.5 mm. The interior passage is all on the glide sheet 266. The top is sleeve membrane 270. The sleeve 252 includes the tubular polymer membrane 270 that includes arm holes 274 on its proximal end next to peg holes 276 and finally sleeve hook holes 272 at the distal end of the sleeve. The sleeve membrane 270 advantage is it adds extra stretch internally, gives a continuous surface as an instrument is slid into the eye, give a better seal from back flow as the elastic is stretched across its surface, can have some elements on the back side to keep the sleeve in place and hooks on the end to snag the sleeve. The glide sheet comes through the incision under the arch 258. This extension gives the incisional area more width variability as the arch can be bent without limiting the incisional size. If the internal plate extended into the incisional area the opening will be limited by the width of the plate. Instruments will be guided into the eye by the externalizing of the plate and will not catch or snag the sleeve which envelopes the plate. A “V” shape is desirable with the V being wider internally.

The protector may have multiple sizes intended for different-sized incisions as required in different procedures. The dimension of the sleeve portion of a protector may be 1.0 mm to any width and length required to encompass the width and depth of an incision. In larger forms, the protector may have width and length dimensions of from about 3 mm to 5.5 mm, or alternatively about 2.6 to 3.5 mm. In smaller versions of the device, the width and length dimensions may be from about 1 mm to 2.5 mm, or alternatively about 1.5 mm to 2.5 mm. In all examples, a sleeve may be sized so that the expansion of the sleeve after insertion into an eye can create a watertight seal to the eye for infusion or extraction of fluid into and out of the eye.

Surgical glove materials are a good material to form a sleeve because of their elasticity and resistance to tearing. An arch or the other mechanisms for expansion described will stretch the sleeve portion to create the seal and protection of the tissues in the passage into the eye. The internal, distal part of the sleeve expands out further than the external, proximal portion of the sleeve adding additional sealing and reducing lateral locking of motion of the instruments working in the eye. The internal crossmember will add additional stretch internally to aid in creating a seal and will also push the iris down under the incision. This further reduces the likelihood of expulsion of iris out of the incision.

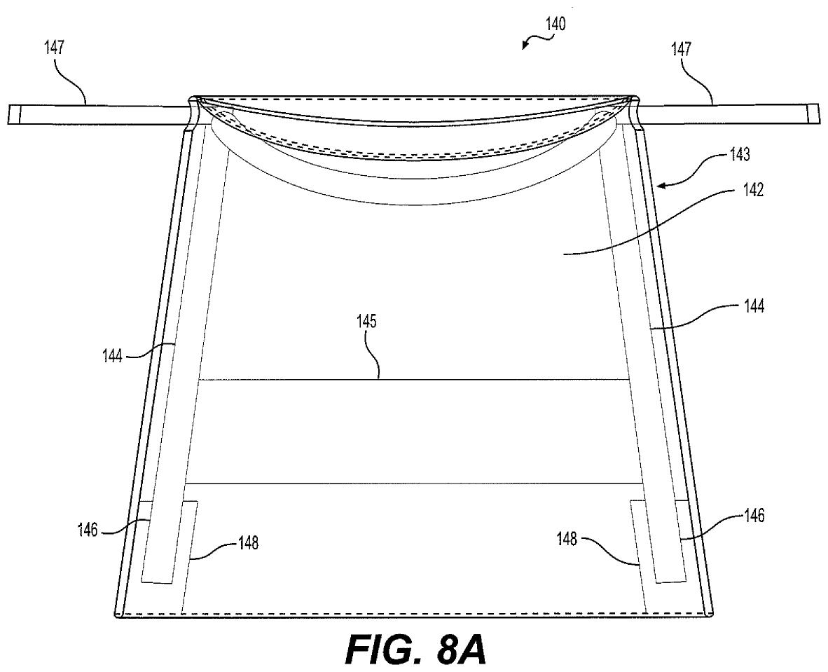

As shown in FIG. 8A, a sleeve 142 may have side pockets 148 therein to receive the arms 144, and specifically the distal end 146 of the arms into to secure the inner, distal aspect of the sleeve 142 and to create a seal from the lateral stretching. The sleeve 142 will pass over the wings 147 of the frame 143 for securing the sleeve externally over the arms 144. A sleeve may alternatively have a loop of material to fold over the arch and back into the sleeve. In another example of a protector 150 as shown in FIG. 8B, a small clip 158 may be used to secure the sleeve 152 to the arms 154, and specifically the distal end 156 of the arms. As the internal crossmember 145 and 155 is in the tunnel, it will be shaped as a flat wedge to allow instruments to glide over it without snagging on a square edge. The C shaped clips 158 may be inserted over the sleeve 152 to clip it to the arch portion (not shown) externally and to the arms 154 internally as necessary to prevent sleeve motion. A tongue and groove with the C clip 158 will keep the sleeve 152 from sliding on the arm 154 or off the arch when instruments are sliding into and out of the eye. Still further alternately, the sleeve may be heat sealed to the arms and the arch of the device.

The sleeve can be lubricated with viscoelastic. As the sleeve will be used for thrusting instruments into the eye to reduce friction inside the sleeve may aid in maintaining the sleeve and giving free movement to the instruments being used through it. Some materials like silicone become slippery when wet, so no additional lubrication may be needed. On the other hand, using a small amount of hyaluronate would facilitate smooth movement. This substance is used routinely at the beginning of cataract surgery to fill the eye so a thin line of it left inside the sleeve would aid in passing instruments during the procedure.

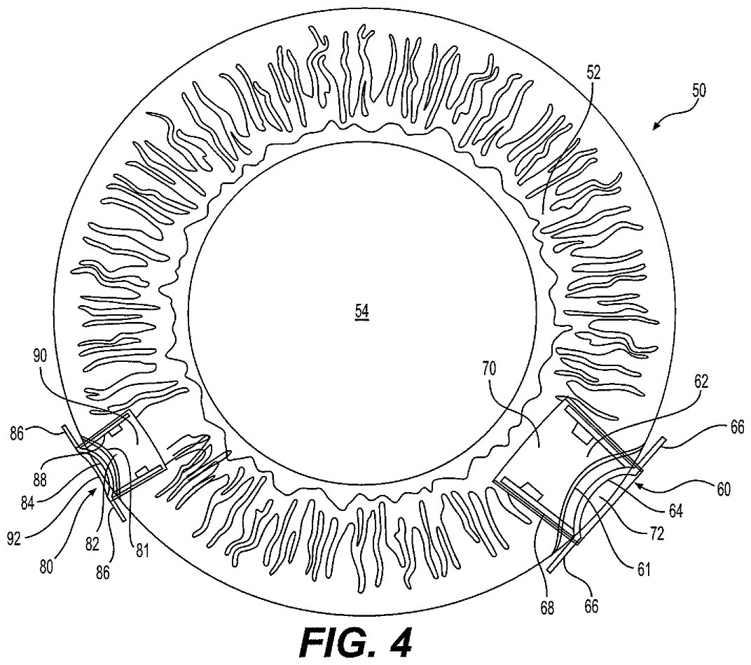

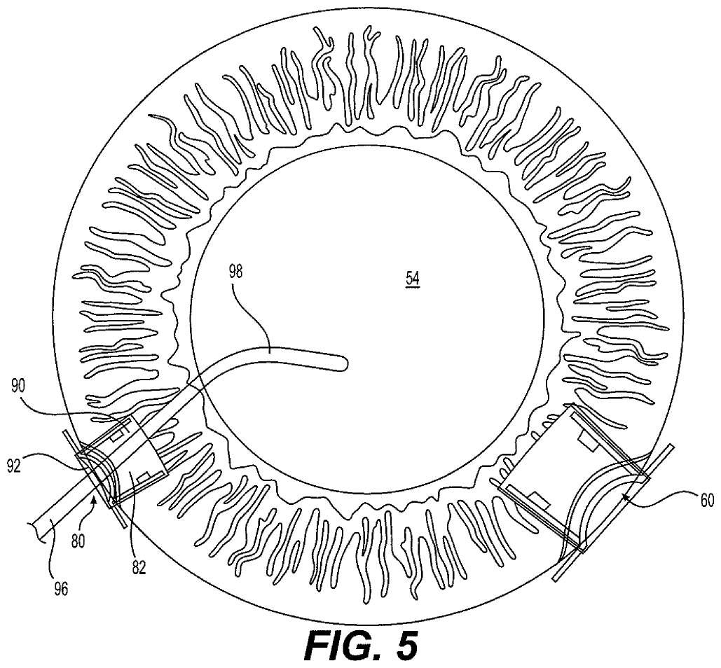

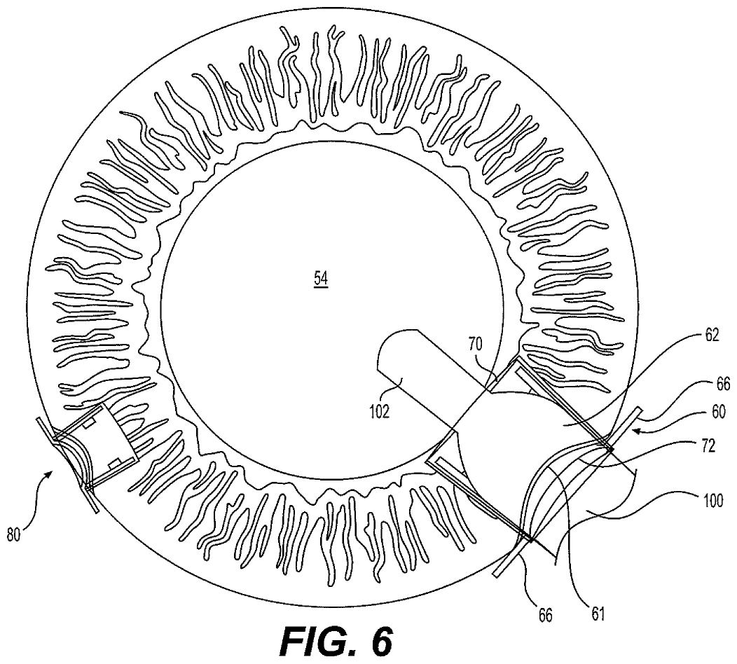

Examples of the use of a protector such as protector 10 are shown in FIGS. 4-6. In FIG. 4, protectors 60 and 80 are shown positioned in eye 50 over the iris 52 thereby forming a passage to or near a cornea 54. As is apparent, protector 60 is larger in width than protector 80. In common parlance, protector 60 is installed in the main incision 61, while protector 80 is installed in smaller incision 81—incision 81 is often referred to as a side port. Protector 60 has a frame comprised of arms 68, wings 66, and an arch 64. A cross member is not visible, because it is covered by the sleeve 62. The wings 66 extend sideways beyond the width of incision 61 and prevent the protector 60 from being inadvertently pushed into the eye 50. The sleeve 62 has an opening 72 on the proximal end of the sleeve and an opening 70 at the distal end of the sleeve. Protector 80 is similar to protector 60 only smaller in width for a smaller incision 81. Protector 80 has arms 88, an arch 84 and wings 86. The wings 86 extend outwardly farther than the incision 81 to secure and prevent the protector from being pushed into the eye 50. Sleeve 82 has an opening 92 on its proximal end and an opening 90 in its distal end. FIG. 4 does not show any medical instruments passing through the sleeves 62 and 82. In FIG. 5, a curved tool 98 is shown passing through the sleeve 82. The surgeon would use the handle 96 to manipulate the tool 98 in the eye. Similarly, another tool 102, a phacoemulsifier, is shown passing through the sleeve 62 into the eye. The handle 100 of the tool 102 is used by the surgeon for work in the eye. In both examples of FIGS. 5 and 6, the operating tools are guarded from damaging the eye tissue by passing through the sleeves 82 and 62 respectively.

FIGS. 17-19 illustrate another example of a protector 280 including frame 250 engineered to be sued in connection with a sleeve 252 to provide a hollow tubular path to the inside of an eye for various procedures. The frame 250 includes a pair of arms 254 connected on their proximal end to wings 256. The arms 254 are also connected on their proximal ends to an arch 258 that secures the arms together. The arch 258 has small spikes 260 extending upwardly from the arch. The distal end of the arms 254 include sleeve securing hooks 264 that extend laterally outwardly from the arms. Also positioned between the arms 254 is a glide plate 266. Sleeve 252 is comprised of a hollow tubular sheet 270 formed of an elastic and flexible material that may be stretched over the frame 250. The tubular sheet 270 has holes 272 on a distal end thereof that receive and are secured in the sleeve securing hooks 264. On the proximal end of the tubular sheet 270 there are two pairs of holes 274 and 276. The holes 274 are adapted to be stretched over and then secured around the wings 256. The holes 276 are adapted to receive pegs 262 that additionally anchor the protector 280 to an eye during use. The tubular sheet 270 is stretched on its proximal end so that it lays on and is secured to or punctured by the spikes 26 to secure the sheet to the arch 258. Finally, as seen in FIG. 19, the bottom of the glide plate 266 has small prongs 280 that also help secure the tubular sheet 270 around the frame 250. In use, the arms 254 of the protector 280 may be manually squeezed by a surgeon to allow the distal end of the protector to be inserted through an incision in an eye. Then, the arms 254 are released, and the protector will expand in the eye to establish the safe passage through the eye layers.

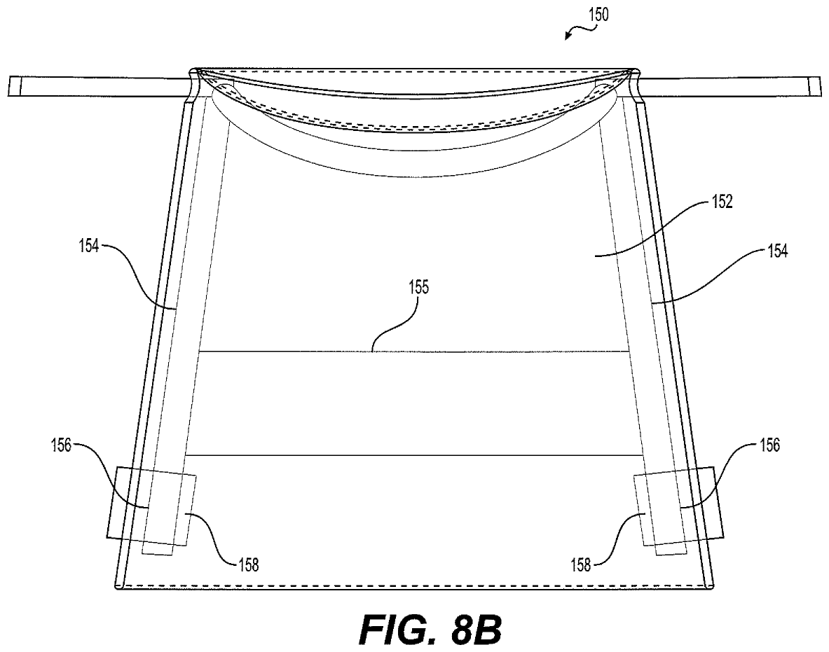

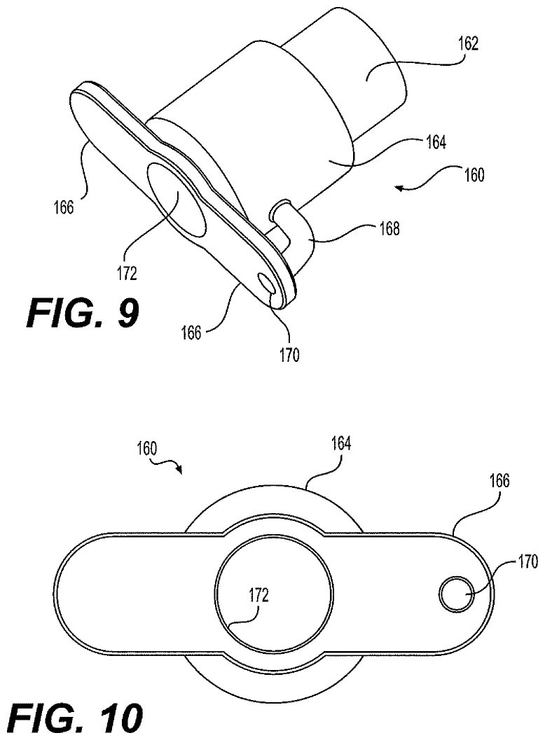

A second general example of a protector is now described in FIGS. 9-16. In this example, a tube made of semi flexible material will be inserted into the incision. The inner or distal end of this tube has a valve to seal out flow but allow instruments to pass in and out of the eye. The valve could be a simple collapsing of the inner aspect of the tube. A “fish mouth” single slit or a bicuspid, tricuspid or quad-cuspid valve could be used. Since the tube material is expandable beyond the bladder area the tube may be closed until opened by pushing an instrument through the valve. The area sealed will start at the end of the bladder area or closer to the inner opening based on function. The use of a plastic or metal valve could be attached to the tip which would push open on a hinge. This hinged system could be used externally as well with a valve that is at the mouth of the tool.

Surrounding this tube is a thin “bladder” with a connecting tube and one-way valve. When the tube is inserted in an eye, the bladder is then filled with saline or hydrogel or air to expand and seal the tube into the incision. The outside of the bladder may have ridges, barbs, spike like projections or other texture shapes to reduce slippage. The expansion of the wall of the membrane would be accomplished using a one-way valve and a cannula to fill and remove filler to the wall of the device. The bladder surrounding the tube could be thin and add minimal width to the incisional width. Not inflated, the bladder may add 0.1 mm. The working passage tube must be stiff enough to resist the expansion of the bladder in the area passing through the cornea. The inner aspect must be flexible to allow the collapse of the tip or the movement of the valve leaves. As incisions are often variable in size the expansion of the bladder would be large enough to encompass a range of incision sizes and the tube portion would be the same for every case. The tube may be wedged shaped to allow for it to secure itself with minimal to no bladder inflation. The system could be used for the main incision and would encompass the size of the “phaco” probe and the IOL tube, in the example of a cataract procedure. Externally, the inner stiff tube will extend laterally to create wings to give support and resist the device being pushed into the eye and when held the device pulling out of the eye. Alternatively, the inflation tube may be crimped into a slit in the wing portion of the device. These types of protectors can be trocar systems. In this iteration, the tip of the trocar for insertion is a sharp cutting shape to allow the tube to be inserted through the cornea. Once inserted the cutting sleeve is removed and the expansion bladder is employed

The material used will be polyamide, poly methyl methacrylate, natural rubber as well as numerous specialty materials like Poron or Neoprene and EDPM. The optimal expansion and resistance to tearing will dictate the ideal material. The bladder system can use latex, rubber, or other expansile materials with surface scaling or pointed or rough elements to reduce slippage. A fabric material can be molded or glued to the surface of the bladder that could allow for the roughed elements to be pushed into the tissue by bladder expansion. The external aspect of the tube will be secured by a ring with two side supports extending on either side of the tunnel device. The inflation valve may be attached to these elements to keep it out of the way of the tunnel area. Alternatively, a slit may be used to crimp the inflation tube eliminating the need for a valve or being used in addition to the valve.

The inner lip of the device will collapse when instruments are removed from the eye. The inner lip may have a tongue and groove shape as necessary to create a watertight seal. The stretching of the sleeve itself will resist back flow of water. To enhance this effect however an enhanced edge that allows one side to fold into the other could further increase the resistance of outflow.

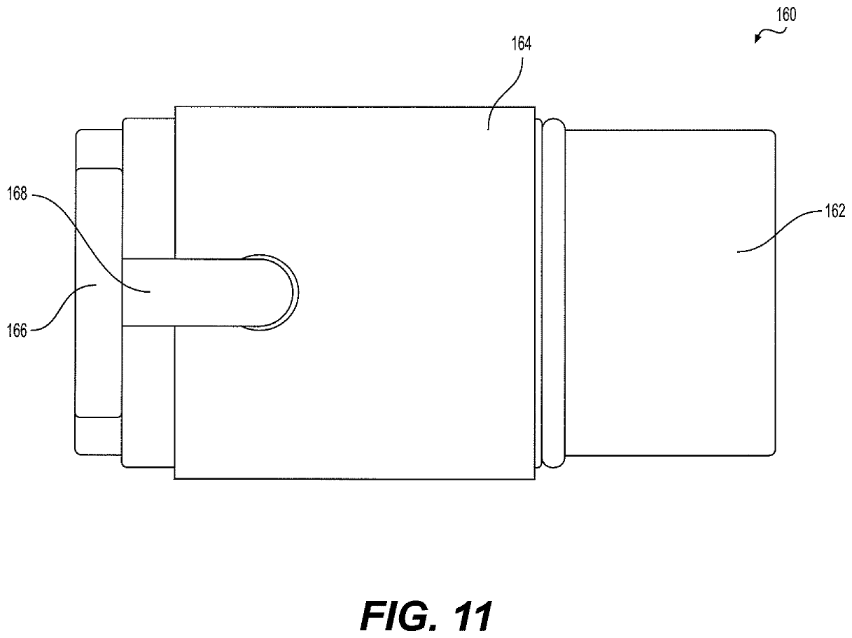

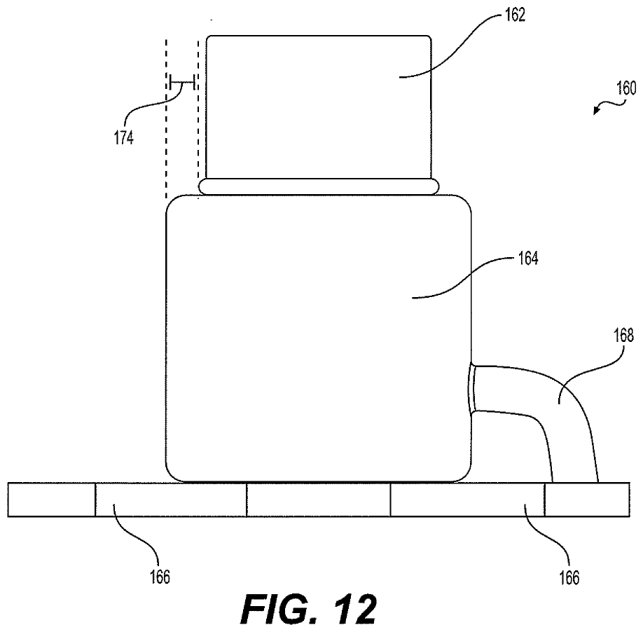

FIGS. 9-12 illustrate one version of this tubular sleeve example. The protector 160 is comprised of a central tubular sleeve 162 that has a bladder 164 formed around all or a portion of the tube. Wings 166 are formed in what will be the proximal end of the protector 160. An opening 172 is the space in the sleeve 162 where instruments will pass through. A cannula 168 connects a valve 170 to the inside of the bladder 164 so that a surgeon will have access to the bladder filling and draining at all times on the proximal end of the protector 160. In FIG. 11, the bladder 164 is shown in its generally flat and uninflated position. In FIG. 12, the bladder 164 is shown in its extended or inflated position. The bladder 164 extends so that its radius is larger by a distance 174 as compared with the uninflated condition of FIG. 11.

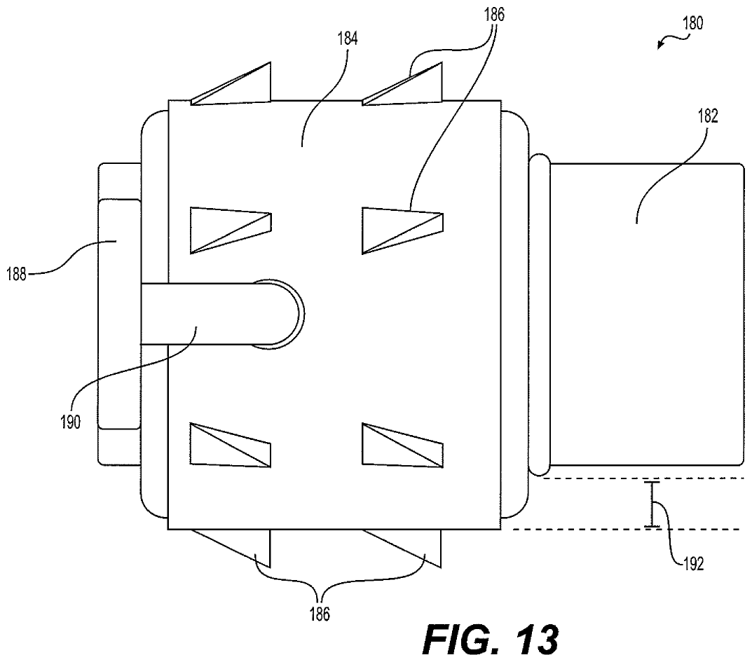

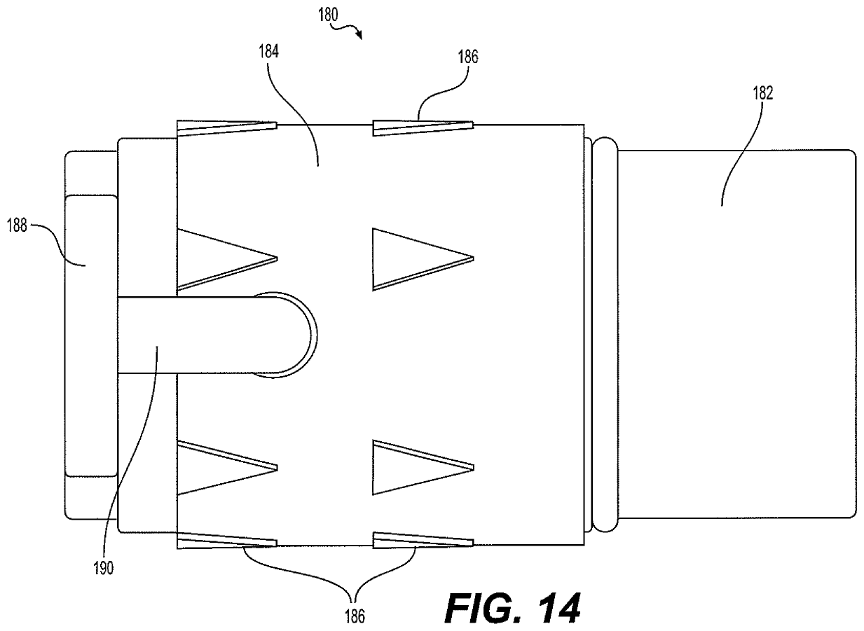

FIGS. 13 and 14 illustrate another version of the tubular sleeve example. There is shown a protector 180 with a tubular sleeve 182 and wings 188. A bladder 184 wraps around about a little more than half the length of the tubular sleeve 182. FIG. 14 illustrates the bladder 184 in its uninflated condition with generally smooth sidewalls, while FIG. 13 illustrates the bladder 184 in its inflated condition that includes barbs 186 that protrude upon inflation. The barbs 186 and extended bladder 184 allow the protector 180 to be secured in place upon installation in an eye. The bladder 184 is connected to a cannula 90 and a valve (not shown) to allow the bladder to be filled and emptied by a surgeon on the proximal end of the protector 180. FIG. 13 shows a length 192 of the extension of the radius of the inflated bladder 184 versus the uninflated condition in FIG. 14.

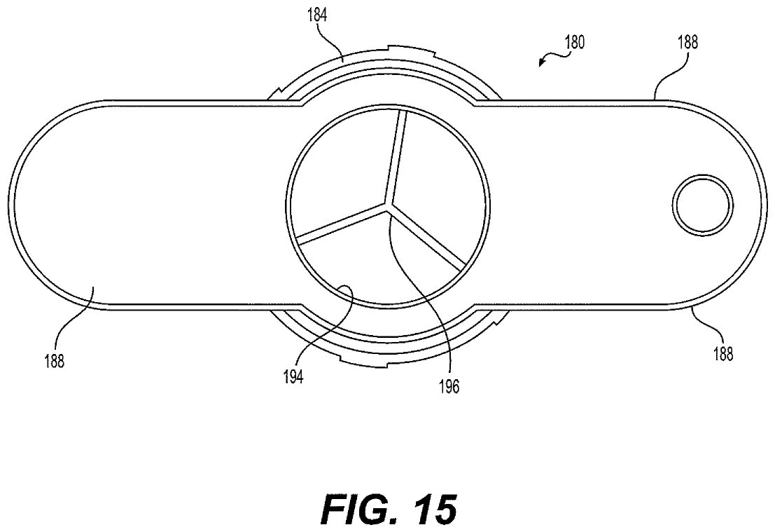

FIG. 15 is a front view of protector 180 having a bladder 184 shown in an uninflated condition, wings 188 and an opening 194. The opening 194 is the beginning of the open passage in the sleeve. The opening 194 is sealed by a tricuspid valve 196, in this example. The valve 196 is resealable and prevents the outflow of eye fluids or materials.

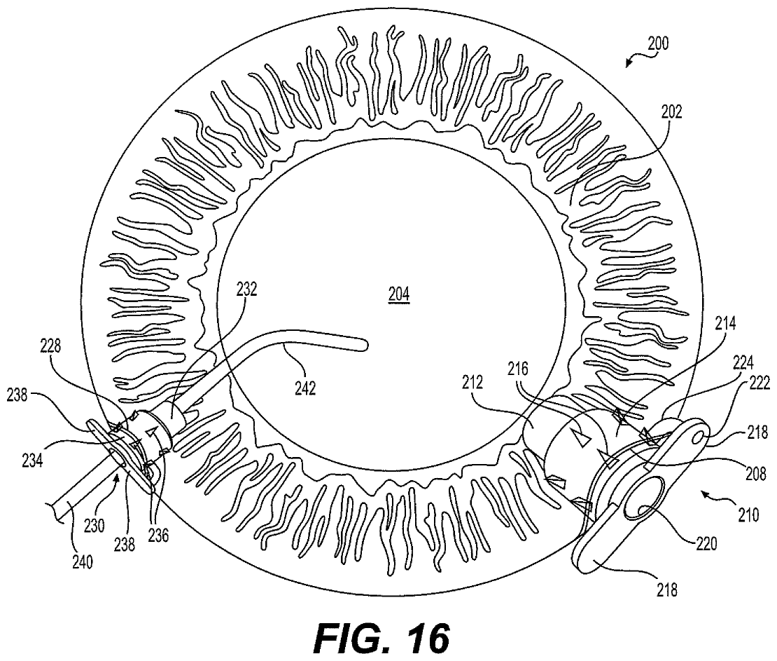

FIG. 16 illustrates two protectors 210 and 230 placed in incisions 208 and 228 in an eye 200 with an iris 202 and cornea 204. Protector 210 has an opening 220 and wings 218 on its proximal end. Also on the proximal end of the protector 210 is a valve 222 and cannula 224 that provide access to the inside of bladder 214. Tubular sleeve 212 is shown with the bladder 214 wrapped around about half of it. Barbs 216 on the outside of the bladder 214 are also shown to help anchor the protector 210 in the incision 208. In this FIG. 16, there are no instruments shown passing through the sleeve 212. Protector 230 is shown installed in incision 228. Protector 230 is dimensionally smaller than protector 210, because protector 230 is positioned in a smaller incision. Protector 230 includes wings 238 and a bladder 236 positioned around the tubular sleeve 232. In this example also, the bladder 234 has barbs 236 that extend outwardly from the inflated bladder surface to additionally secure the protector 230 in the incision 228. An example of a medical instrument tool 242 and handle 240 are shown passing through the protector 230 and into the inside of the eye 200.

The sleeve in all of the foregoing examples may be colored. As visibility through the sleeve tunnel may be helpful in working under the incision, a clear material or minimally opaque or colored is preferred, but as the sleeve and tube are not in the surgical plane they will not block the view in the working area. As the sleeve may be difficult to see through the cornea in the eye and surgeons may wish to monitor that it is in place and functioning well a slight tint would aid in observing any defects as necessary. The bladder filler could be given a color dye as well to aid in visibility and to show its existence and volume.

In another example, the sleeve such as sleeve 12 or 132 may be coated with an antibiotic coating. Alternatively, a sleeve such as sleeve 12 or 132 may be coated with an anti-inflammatory medicine. In each case, the coating is not separately shown in the figures, except that it is coated onto the outside surface of those sleeves. In this manner, the coating may prevent or reduce the chance of swelling or infection after a procedure.

To get a second instrument in the eye in most procedures, a smaller incision of 1.0 mm to 1.5 mm is made about 90 degrees away from the primary incision. The smaller 1-2 mm protector used in a side port may, in some examples, have an inflatable sleeve to secure an infusion line or a pressure monitor. Placing the protector and then inflating it would secure the protector and surgical devices extending there through to the cornea. As the protector provides a sealed access to the anterior chamber in the eye, it could be used to monitor, sample, control and medicate the eye. This can be done through the smaller version inserted through the side port. A port could then be used to infuse and sample the anterior chamber. In cases of uncontrolled IOP it could act as a temporary shunt. In a case of endothelial graft air or gas could be infused. In a case of infection, the anterior chamber could be sampled and infused with antibiotics. In a case of hypotony or low pressure an infusion to maintain adequate IOP could be considered. In normal eye surgery, all of these methods would assist in providing a data source and a method for controlling the infusion of meds which could include dilation meds. The device could be used with a Trocar-like internal blade to place the tube and then be secured by the bladder inflation and the slide resistant elements on the side of the tube.

A hard plastic or soft external valve can be used for this device. The valve in this case would be enclosed in the external piece. The valve could be a hinged system with a plastic or metal valve. A ball valve that is displaced by insertion of a tool through the tube. A collapsible material like rubber, sponge or other elastic materials could seal tube until driven open by an inserting instrument.

This protector may be designed to inflate within the incision to provide a sealed incision while allowing access to a central passage for infusion of fluid into the eye or extraction of fluid out of the eye. The device could use the central tube to place a probe into the eye for exact measurement of intraocular pressure in real time. The water pressure expressed out of the tube could be measured with an external manometer. The IOP could be adjusted with feedback to an infusion pump. This could be used intraoperatively to place an implant without using visco elastic, to determine that the IOP is normal and that the wounds are watertight at the conclusion of the surgery. They could be used to maintain a normal pressure in a case of uncontrolled IOP crisis until another intervention could be used. The same protector could be placed through the sclera to measure the intravitreal pressure.

Other embodiments of the present invention will be apparent to those skilled in the art from consideration of the specification. It is intended that the specification and figures be considered as exemplary only, with a true scope and spirit of the invention being indicated by the following claims.

That which is claimed is:

1. A protector for a trans-corneal incision comprising:
a tubular sleeve that defines a width and length, wherein the sleeve has a proximal end and a distal end;
wherein the proximal end includes wings that extend outwardly from the width of the sleeve, the proximal end adapted to engage the perimeter of an ocular incision and the wings extend outside of the perimeter of the ocular incision to prevent the sleeve from being pushed into an eye;
wherein the distal end is adapted to extend into intraocular eye space in the direction of the length of the membrane.

2. A protector for a trans-corneal incision as described in claim 1,
wherein the tubular sleeve is a flexible membrane.

3. A protector for a trans-corneal incision as described in claim 2,
further comprising a frame that is connected to the sleeve and holds the sleeve in an expanded position to secure the sleeve in an eye.

4. A protector for a trans-corneal incision as described in claim 1,
wherein the sleeve further comprises a plurality of small spikes that project outwardly from the outside of the sleeve for securement of the sleeve in an intraocular incision.

5. A protector for a trans-corneal incision as described in claim 1,
wherein the tubular sleeve comprises a rigid tube.

6. A protector for a trans-corneal incision as described in claim 5,
wherein the rigid tube has an expandable bladder mounted around an outside portion of the rigid tube.

7. A protector for a trans-corneal incision as described in claim 1,
wherein the sleeve is colored.

8. A protector for a trans-corneal incision as described in claim 1,
wherein the sleeve is clear.

9. A protector for a trans-corneal incision as described in claim 3
wherein the frame comprises two arms connected by an arch, and the arch comprises small spikes projecting outwardly therefrom to secure to the flexible membrane.

10. A protector for a trans-corneal incision as described in claim 1,
wherein the distal end of the sleeve has a larger width dimension than the proximal end of the sleeve to aid in lateral movement of instruments and to resist extrusion of the sleeve through the incision on withdrawal of instruments.

11. A protector for a trans-conical incision as described in claim 1,
wherein the sleeve is coated on its outside surface with an antibiotic material.

12. A protector for a trans-corneal incision as described in claim 1,
wherein the sleeve is coated on its outside surface with an anti-inflammatory material.
